(19) United States
(12) Patent Application Publication
(10) Pub. No.: US 2026/0153447 A1
(43) Pub. Date: Jun. 4, 2026

(54) SENSING ASSEMBLY FOR A GAS SENSOR FOR DETECTING A REACTIVE GAS

(71) Applicant: United Fiber Sensing Holding B.V., THE HAGUE (NL)

(72) Inventor: Korina Luise Frederike HARTMANN, THE HAGUE (NL)

(73) Assignee: United Fiber Sensing Holding B.V., THE HAGUE (NL)

(21) Appl. No.: 18/867,684

(22) PCT Filed: Jun. 22, 2023
(86) PCT No.: PCT/NL2023/050346

§ 371 Date: Nov. 20, 2024

(30) Foreign Application Priority Data
Jun. 23, 2022 (NL) 2032267

(51) Int. Cl. G01N 21/77; G01D 5/353; G01K 1/02; G01K 11/3206; G01N 33/00

(52) U.S. Cl. CPC G01N 21/774; G01K 1/026; G01K 11/3206; G01N 33/0031; G01N 33/005; G01D 5/35316; G01N 2021/7716; G01N 2021/773; G01N 2021/7773

(57) ABSTRACT

A sensing assembly for detecting a reactive gas to be sensed such as hydrogen, a method of manufacturing thereof, and a gas sensor. The sensing assembly includes an optical fiber having a temperature sensitive detection zone, and a reaction layer associated with an external surface of the optical fiber positioned at least adjacent to the temperature sensitive detection zone. The reaction layer includes a polyimide-based polymer and a reactive composition adapted to generate a temperature change when brought into contact with the gas to be sensed. The temperature sensitive detection zone may be a fiber Bragg grating. The reaction layer may include the polymer. The reaction reactive composition may include tungsten oxide and a catalyst such as platina.

The present patent disclosure relates to a sensing assembly for detecting a reactive gas to be sensed such as hydrogen, a method of manufacturing a sensing assembly for detecting a reactive gas to be sensed such as hydrogen, and a gas sensor.

Gas sensing or detection can be done in various ways, depending on the gas to be sensed. For reactive gases such as hydrogen, fiber optic based sensors exist that have a temperature sensitive detection zone such as a fiber Bragg grating combined with a reactive compound that, when hydrogen reacts at the reactive compound, cause the temperature sensitive detection zone to heat or cool in case the reaction is exothermic or endothermic, respectively. When this zone changes temperature, one or more optical properties of that zone in the fiber change, such that the light passing through this zone is altered in a manner that can be detected. The advantage of these types of sensors is that there is no electrical contacts near the location where the reactive gas is actually sensed. In this way, electronic discharges are reduced and therefore the safety of the sensor equipment is enhanced for flammable/explosive gases such as hydrogen.

A fiber Bragg grating (FBG) is a type of distributed Bragg reflector in a segment of the optical fiber that reflects particular wavelengths of light and transmits all others. This is achieved by creating a periodic variation in the refractive index of the fiber core, which generates a wavelength-specific dielectric mirror. When the temperature in the zone or segment with the fiber Bragg grating changes, the wavelength of the light that is reflected changes. This may, for instance, be caused by an elongation/expansion or shortening/contraction of the fiber core and an accompanying change in the periodicity of the FBG. The change in the wavelength of reflected light can be detected with a light detector e.g. by detecting the change in wavelength using a spectrometer or by detecting a one or more changes in amplitude at one or more certain fixed wavelength or wavelength bands.

It is possible to create multiple FBG's along a single optical fiber, or a chain of connected optical fibers. When each FBG has its own specific reflected wavelength (band), it is possible to use a single light source and detector for multiple gas detection zones at the FBG's.

Patent publication WO 2008/125686 A1 concerns a gas sensor comprising sensing assembly including a fiber having a fiber Bragg grating segment. The FBG segment is surrounded by a reaction layer comprising a combination of tungsten oxide (WO₃) nano or micro powder and a catalyst such as platina (Pt). When the reaction layer is exposed to a certain concentration of hydrogen, the hydrogen reacts to form water at/in the reaction layer and heat is produced by this reaction, thereby heating up the fiber Bragg grating segment. This reaction reaches an equilibrium state and therefore the reaction layer will be heated to an equilibrium temperature. The change in the wavelength of the reflected light is directly related to the hydrogen concentration of the surrounding gas mixture. These types of reaction layers usually have a length in the order of centimeters, as longer layers will general break and/or fall off due to the mechanical properties of the mixture of tungsten oxide powder and catalyst.

It is an object, among objects, of the present patent disclosure to provide improved sensing assemblies and gas sensors.

According to a first aspect, there is provided a sensing assembly for detecting a reactive gas to be sensed such as hydrogen, the sensing assembly being for use in a gas sensor, the sensing assembly comprising an optical fiber having a temperature sensitive detection zone; a reaction layer associated with an external surface of the optical fiber positioned at least adjacent to the temperature sensitive detection zone, wherein the reaction layer comprises a polyimide-based polymer and a reactive composition adapted to generate a temperature change when brought into contact with the gas to be sensed.

The above described sensing assembly including a reaction layer with a combination of polyimide-based polymer and the reactive composition has several advantages. These advantages include that the reaction layer has an increased mechanical stability, and therefore longer lengths of optical fiber can be made with the reaction layer. A further advantage is that the applying of the reaction layer is made easier, as a liquid of the polyimide-based polymer with the reactive composition can be applied to the outside of the optical fiber.

In addition to this, the reaction layer including a polyimide-based polymer is especially beneficial because optical fibers with polyimide-based polymer fiber coating, which are heat resistant and non-reactive, are readily manufactured using techniques such as a draw tower. This enables the manufacturing of long lengths of the sensing assemblies described above, thereby enabling measuring of reactive gas concentration in the surroundings in a continuous manner along the fiber length. In this way, the sensing assembly can be used as a novel distributed reactive gas sensor, based on distributed temperature sensing techniques known in the art. It is preferred that the optical fiber comprises the reaction layer continuously along at least a length of 0.3 m, preferably at least 1 meter, optionally up to substantially the whole length of the optical fiber. Typically, in the field, a sensing assembly used as distributed reactive gas sensor would have a length in the order of tens of meters up to hundred kilometers or more.

The sensing assembly including the polyimide-based polymer provided in the sensing assembly also causes a reduced reaction rate at the reactive composition, which in turn causes the temperature sensitive detection zone to experience a smaller change in temperature. In this way, the change in optical properties at the temperature sensitive detection zone are reduced, and thus the required bandwidth of a light source and/or a light detector in the gas sensor device including the sensing assembly is reduced. In this way, with a single light source having the same bandwidth, more sensing assemblies can be measured at the same time than for known gas sensors employing temperature sensitive detection zones. Alternatively, the same result is achieved with a sensing assembly having one or more fibers with multiple temperature sensitive detection zones.

An additional benefit due to the smaller change in temperature, is that the reaction layer itself also experiences less mechanical stress, and thus the lifetime is increased. Furthermore, the safety of the sensing assembly, when in use in a gas sensing device, is increased in particular when the reaction is exothermic, since the temperature changes are associated with a smaller energy input and thus there is a smaller chance of the reactive gas to be sensed to burn or explode.

A further advantage of the use of the polyimide-based polymer is that the reactive layer is more flexible, and thus less brittle, such that the reactive composition is mechanically stabilized on the fiber.

Polyimide provides the technical advantage of being heat resistant and chemically inert. In addition, polyimide is used as fiber coating in heat resistant optical fibers and therefor existing processes can be used to coat the fiber with the reactive layer, since in this case a mixture of polyimide-based polymer and the reactive composition can be used.

In an embodiment, the reaction layer comprises the polymer causing a reduced reaction rate at the reactive composition. The polymer may be configured to reduce the rate of diffusion of the reactive gas towards the reactive composition, such that the reaction rate at the reactive composition is lowered and thus the temperature change experienced by the temperature sensitive zone is reduced. Additionally or alternatively, the polymer may provide a thermal barrier thus reducing the amount of heat transferred to (in case of an exothermal reaction) or from (in case of an endothermic reaction) the temperatures sensitive zone, again causing a reduction in the experienced temperature change by the temperature sensitive zone. The reactive composition may be provided at or near the surface of the polymer.

It is preferred that the polyimide-based polymer is polyimide.

Preferably the reaction layer comprises polyimide in an amount of at least 10 wt %, preferably in the range of 20-100 wt %, relative to the reactive composition. More preferred the reaction layer comprises polyimide in the range of 20-60 wt %, most preferred 25 wt % or more, for instance 30-50 wt %, relative to the reactive composition. These ranges result in a particularly stable and scratch-resistant coating, especially when combined with tungsten oxide with platina catalyst as the reactive composition.

In an embodiment, the polyimide-based polymer material comprises hydrophobic monomers. The hydrophonic monomers are preferably hydrophobic fluorinated monomers. The hydrophobic fluorinated monomers may be selected from the group consisting of 2,2-bis(3-amino-4-hydroxyphenyl) hexafluoropropane, 2,2-bis(3,4-dicarboxyphenyl) hexafluoropropane dianhydride (6FDA), bis(trifluoromethyl)benzidine, and 1,4-bis(4-amino-2-trifluoromethylphenoxy)benzene or a combination thereof. The polymer may comprise the hydrophobic monomers in the range of 10-80 wt % relative to polyimide, preferably 20-40 wt %. This preferred weight percentage is particularly applicable when the polyimide polymer material comprises only one type of hydrophobic monomer.

Additionally or alternatively, in an embodiment the polyimide-based polymer material is or comprises a copolymer of polyimide with one or more of polysiloxanes, polyvinylidene fluorides, polytetrafluoroethylenes, and polyetherimides. Preferably the polyimide-based polymer material comprises 10-50 wt % of the one or more of polysiloxanes, polyvinylidene fluoride, polytetrafluoroethylene and polyetherimides relative to polyimide.

The use of hydrophobic monomers or components in combination with the polymer results in avoiding water absorption in the polymer, which may result in an expansion of the polymer and thus a concomitant expansion of the fiber at the temperature sensitive detection zone such as a FBG, reducing false positive and/or negatives.

In an embodiment, the reactive composition comprises tungsten oxide or vanadium oxide, and a catalyst selected from the group consisting of catalytic metals and catalytic metal oxides.

Preferably, the catalyst is platina and/or the reactive composition comprises tungsten oxide. When combined, the tungsten oxide can be doped with platina. The tungsten oxide and platina are suitable for use with the polyimide-based polymer material.

Further embodiments are defined in the dependent claims, the advantages of which will become apparent from the below description of figures.

According to second aspect, there is provided a method of manufacturing a sensing assembly for detecting a reactive gas to be sensed such as hydrogen, the sensing assembly being for use in a gas sensor, the method comprising providing an optical fiber having a temperature sensitive detection zone; applying a reaction layer onto an external surface of the optical fiber, wherein the reaction layer is applied at least adjacent to the temperature sensitive detection zone, wherein the reaction layer comprises a polyimide-based polymer and a reactive composition adapted to generate a temperature change when brought into contact with the gas to be sensed; and providing a polymer causing a reduced reaction rate at the reactive composition of the reactive gas to be sensed.

In an embodiment, the applying of the reaction layer comprises providing a mixture comprising a polyimide precursor, the reactive composition and a sacrificial compound decomposing at a decomposition temperature and applying the mixture onto the external surface of the optical fiber, wherein the reaction layer is applied at least adjacent to the temperature sensitive detection zone, wherein the method comprises, after applying the mixture, annealing the sensing assembly at elevated temperature of at least the decomposition temperature such that the reaction layer is formed, wherein the formed reaction layer is porous. The decomposition temperature of the porogen in this case is preferably equal to or higher than the curing temperature of the polyimide. The curing temperature of polyimide is typically in the range of 180° C. and 400° C. Higher temperatures are possible, but then it is preferred to use a nitrogen atmosphere during the annealing, in order to avoid unwanted reactions of the polyimide with oxygen.

Advantageously, the sacrificial compound being sacrificed causes the resulting reaction layer to be porous, thus resulting in an increased kinetics for reactive gases entering the reaction layer as well as reaction products leaving the reaction layer.

According to yet another aspect, there is provided a gas sensor, gas sensing device or gas detector comprising the sensing assembly according to the first aspect or manufactured according to any one of the methods of the second aspect.

Further embodiments of the various aspects are defined in the dependent claims, the advantages of which will become apparent from the below description of figures.

Features, advantages and effects of the various aspects and embodiments are readily applicable to any of the other aspects and embodiments, as will be understood, also by a person skilled in the art.

The accompanying drawings are used to illustrate presently preferred non-limiting exemplary embodiments of devices of the present disclosure. The above and other advantages of the features and objects of the disclosure will become more apparent and the aspects and embodiments will be better understood from the following detailed description when read in conjunction with the accompanying drawings, in which:

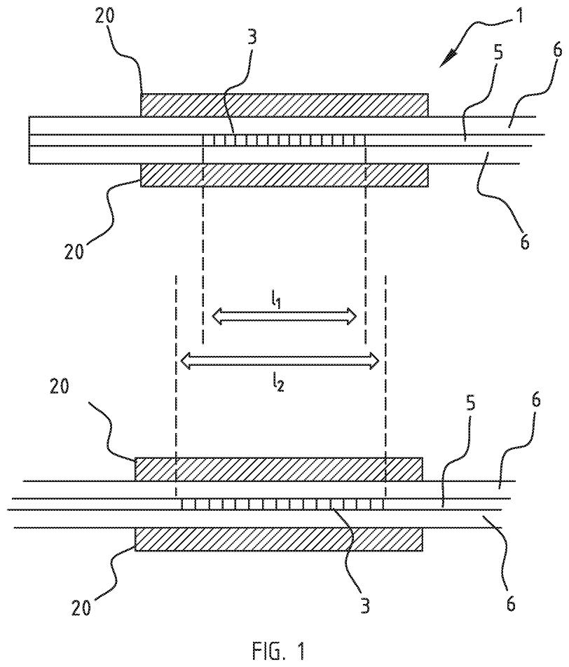

FIG. 1 shows two schematic longitudinal cross section views of a sensing assembly according to the present patent disclosure;

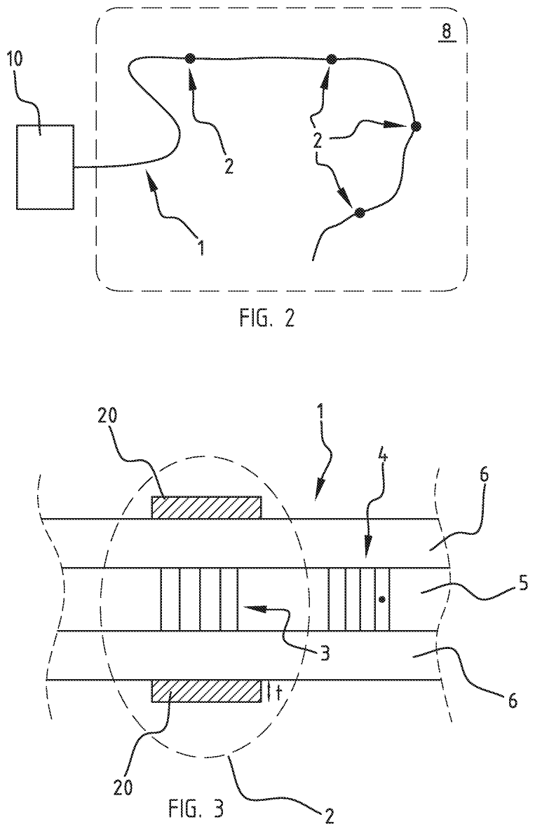

FIG. 2 is a schematic drawing if a gas sensor with sensing assembly according to the present patent disclosure in a region to be monitored;

FIG. 3 is a schematic longitudinal cross section view of a sensing assembly according to the present patent disclosure;

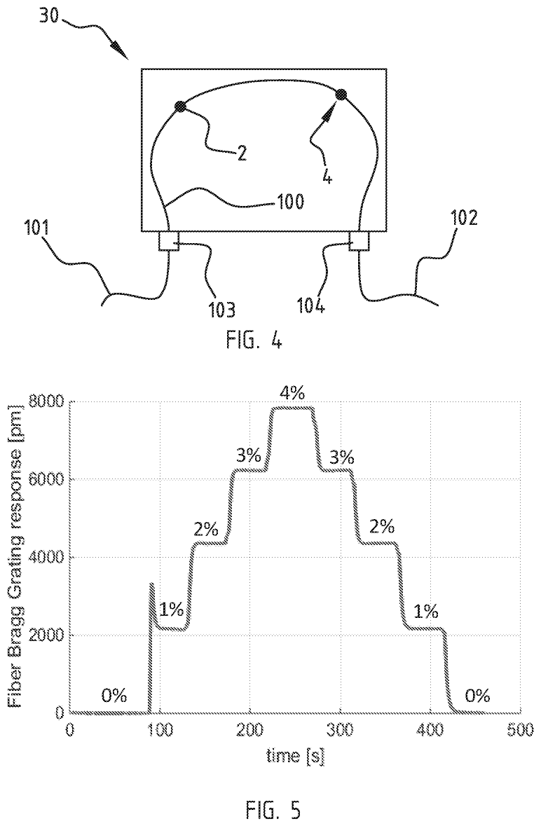

FIG. 4 is a schematic top view of a sensing assembly in a (n open) casing with fiber connections according to the present patent disclosure;

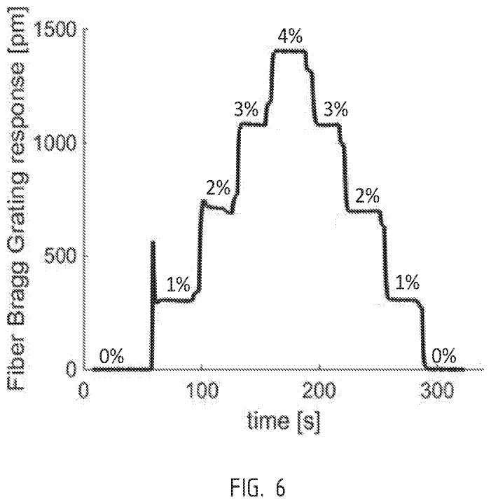

FIG. 5 is a graph showing on the left axis measured wavelength shifts versus time sensed by a reference sensing assembly for various hydrogen concentrations in air; FIG. 6 is a graph showing on the left axis measured wavelength shifts versus time for a sensing assembly according to the present patent disclosure comprising a reaction layer with a polyimide-based polymer material for various hydrogen concentrations in air;

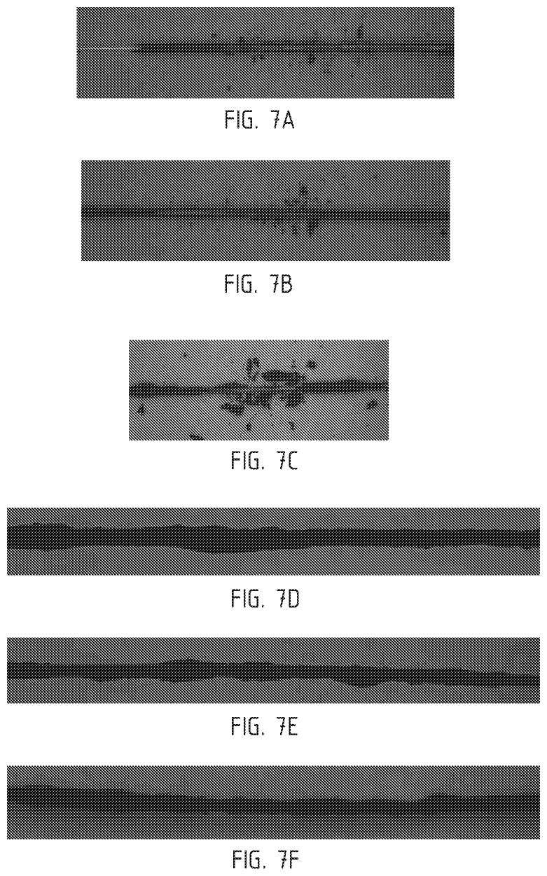

FIG. 7A-F are photos of sensing assemblies comprising optical fibers coated with a reaction layer according to the present patent disclosure comprising a polyimide-based polymer material comprising respectively 7 (FIG. 7A), 10 (FIG. 7B), 20 (FIG. 7C), 30 (FIG. 7D), 40 (FIG. 7E) and 50 (FIG. 7F) wt % polyimide relative to the reactive material, the photos being taken after a scratch test;

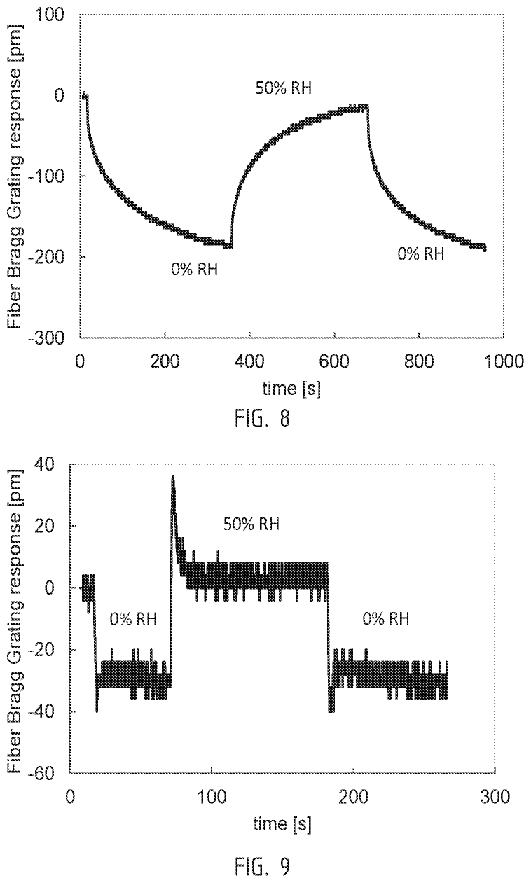

FIG. 8 shows the fiber Bragg grating response versus time for a sensing assembly according to the present patent disclosure comprising a reaction layer with a polyimide-based polymer material for varying relative humidity;

FIG. 9 shows the fiber Bragg grating response versus time for a sensing assembly according to the present patent disclosure comprising a reaction layer with a polyimide-based polymer material comprising hydrophobic monomers for varying relative humidity; and

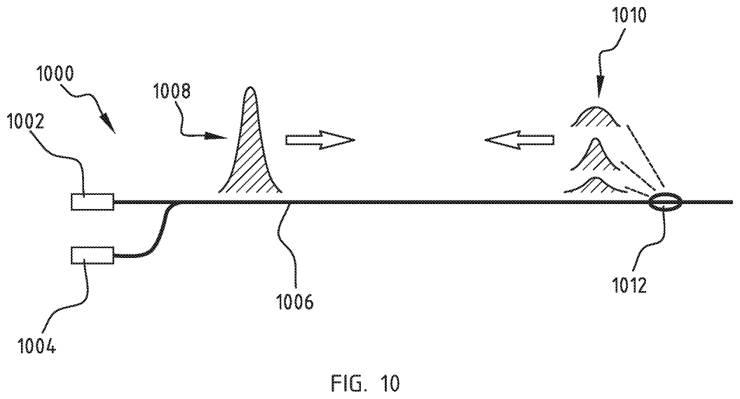

FIG. 10 shows a distributed hydrogen sensor using Raman scattering and an sensing assembly according to the present patent disclosure.

As is shown in FIG. 1, a sensing assembly comprises optical fiber 1, comprising a temperature sensitive detection zone, here embodied as a fiber Bragg grating (FGB) 3 which is patterned into the fiber core 5. The optical fiber also comprises a fiber cladding (not shown) surrounding the fiber core 5 and a fiber coating 6 surrounding the fiber cladding. The fiber cladding is typically a glass layer surrounding the fiber core in order to achieve the total internal reflection of light propagating through the fiber core/cladding. The FBG 3, positioned in fiber core 5, is surrounded by the reaction layer 20 with the fiber cladding in between. The reaction layer 20 comprises the reactive composition which reacts with the reactive gas such as hydrogen. The fiber core 5 is surrounded by fiber coating 6. When there is no hydrogen, the FBG has a temperature typically equal to the temperature of the surrounds and a length 11 as indicated for the top sensing assembly in FIG. 1.

The bottom sensing assembly in FIG. 1 shows the effect of increased hydrogen pressure. The hydrogen diffuses into the reaction layer 20 and the reactive composition reacts with the hydrogen and heat is produced. This heat heats up, among other things, the fiber core 5 which slightly elongates. Thus the FBG 3 also elongates and its periodicity changed. This change of periodicity can be measured by a wavelength shift or peak intensity change using a suitable detector. Since the used reactive compositions react with the reactive gas (hydrogen) in air with an equilibrium reaction, a certain temperature measured in the FBG 3 corresponds to a specific reactive gas (hydrogen) pressure in the air surrounding the sensing assembly

As shown in FIG. 2, a sensing assembly is used in an area/volume to be sensed 8. Although in this example there is shown a single fiber 1 having multiple sensing positions 2, it is also an option that there are multiple fibers having one or more of the sensing positions 2. The multiple fibers may be daisy chained together such that one set of sensing equipment 10 may be used. The sensing equipment 10 comprises a light source and a light detector. The light source emits light into the fiber 1, and a part of the light is reflected at each sensing position 2 by the fiber Bragg gratings (FBG's) 3 (FIGS. 1 and 3). How the FBG's bring about a change in peak position of the reflected light due to a change in temperature in the FBG is well known and explained for instance in patent document WO 2008/125686 A1, which is included by reference in its entirety into the present patent disclosure, see e.g. page 3, line 21 to page 4, line 14.

The light detector may be a spectrometer, but may also be implemented as multiple light sensors specifically configured to measure a peak reflectivity at a specific wavelength. A measure for the reflectivity can be obtained by measuring the intensity of light coming back out of the fiber 1 at the light source in sensing equipment 10. Suitable fiber splitters or other optical elements (not shown) are used to direct only the light coming back out from the fiber towards the light detector.

Each sensing position 2 comprises at least FBG 3, which is surrounded by the reaction layer 20. Each FBG 3 is has a varying periodicity such that each FBG 3 is associated with a specific peak wavelength that is reflected. The light sensors can thus be configured to each measure a peak reflectivity at a respective peak wavelength of the FBG's 3, such that only one light source is required. Also a spectrometer or computer obtaining data of such a spectrometer can be configured to detect the peak reflectivity or light intensity at those peak wavelengths.

The sensing position 2 is shown in an enlarged manner in FIG. 3. The FBG 3, positioned in fiber core 5, is surrounded by the reaction layer 20, which comprises the reactive composition which reacts with the reactive gas such as hydrogen. The fiber 1 further includes reference FBG 4, which is used as a reference to correct for temperature changes of the fiber unrelated to gas reacting in the reaction layer. The reaction layer has a thickness t that can be varied. A thickness of the reaction layer in the order of about 0.5 or 1 μm results in a sufficiently mechanically stable reaction layer. In general, the reaction layer has a thickness of at least 0.5 μm up to 500 μm. It is preferred that the reaction layer thickness lies in the range of 10 μm and 300 μm, more preferably between 18 and 100 μm. It is noted that commercial optical fibers with polyimide coatings have a coating thickness of about 20 μm. Therefore, the suitable ranges of coating thickness of the reaction layer of the present patent disclosure is compatible with these commercial optical fibers.

In an embodiment, as shown in FIG. 4, the sensing assembly comprises fiber 100 with an FBG at sensing position 2 and a reference FBG 4. may comprise in a housing 30. In this embodiment, the sensing assembly comprises a housing 30. The housing 30 comprises optical fiber connections 103 and 104, where connection 103 may be used for the input fiber 101 and connection 104 for the output fiber 102, or vice versa. It will be apparent that a plurality of sensing assemblies can be daisy chained in due to this configuration. The casing 30 may comprise a cover (not shown) suitable open to allow for the reactive gas to be sensed to enter the casing 30.

FIG. 5 shows example data of sensor data obtained for varying hydrogen concentrations in air, measured with a sensing assembly comprising a reactive layer with tungsten oxide and platina as the reactive composition, without polyimide. The reaction layer is prepared as described below. The molar ration of WO₃:Pt is 10:1. The thickness of the reaction layer in this example is about 350 μm. The data shows the fiber Bragg grating response or, in other words, wavelength shift versus time. The change in hydrogen gas concentration can be seen at the jumps in wavelength shift, as indicated in the graph by 1.0%, 2.0%, 3% and 4% hydrogen in air. This shows the fast kinetics of these gas sensors. The peak in the data at around 90 s occurs due to the gas control during the experiment not being perfect, there then being for instance an increase in hydrogen pressure and/or decrease in oxygen pressure. The wavelength shift of about 8000 pm for 4% hydrogen gas is equivalent to a temperature change of about 800° C. in the FBG, such as the FBG 3 of FIGS. 1 and 3.

The equilibrium reaction occurring in the reaction layer is as follows:

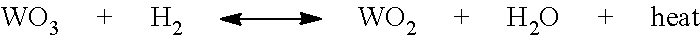

wherein platina acts as catalyst. The ratio of WO₃:Pt in this example is 10:1, but may vary, e.g. 13:1 or 20:1. This would shift the equilibrium so that less heat is produced, thus decreasing the wavelength shift at fixed hydrogen gas concentrations. For instance, when less heat is produced at 2% hydrogen concentration, the wavelength shift will be lower than 4200 μm.

The fiber Bragg grating response of a sensing assembly with a reaction layer with polyimide is shown in FIG. 6. The reaction layer is prepared and applied to the optical fiber as described below. The thickness of the reaction layer in this example is about 200 μm. The molar ratio of WO₃:Pt is 10:1. As shown in FIG. 6 when polyimide is used in the reaction layer 20, the wavelength shift is reduced by over a factor of 5. The wavelength shift at 4% hydrogen is estimated at around 1400 pm, corresponding to a temperature change of 140° C. in the FBG. An advantage of this reduced wavelength shift is that sensing positions can be measured with a single light source having the same bandwidth of light wavelengths, as described above. Further advantages include that lower temperatures are safer and the sensing assembly experiences less thermal stress and thus remains in good condition for a longer period of time.

FIGS. 7A-7F show sensing assemblies with varying amounts of the polyimide-based polymer material relative to the reactive composition, here comprising tungsten oxide including platina as catalyst. The various sensing assemblies comprise respectively 7 (FIG. 7A), 10 (FIG. 7B), 20 (FIG. 7C), 30 (FIG. 7D), 40 (FIG. 7E) and 50 (FIG. 7F) wt % polyimide relative to the reactive material.

The photos being taken after a scratch test with a force of 0.8-1 N using a polypropylene tip. The force is estimated by performing the same procedure on a digital scale and calculating the force from the measured mass. As becomes clear, when 30 wt % or more of polyimide is used, after the scratch test the reaction layer is still intact. In contrast, for the reaction layers with lower amounts of polyimide (7, 10 and 20 wt %) the scratch test results in damaged respective reaction layers.

FIGS. 8 and 9 compare the fiber Bragg grating response (wavelength shift) due to relative humidity for a reaction layer with polyimide (FIG. 8) and a reaction layer with polyimide with increased hydrophobicity due to the inclusion of hydrophobic monomers in the polyimide (FIG. 9). The reaction layer in this example comprised 40 wt % 2,2-bis(3-amino-4-hydroxyphenyl) hexafluoropropane relative to polyimide. The wavelength shift in FIG. 8 is about 200 pm when changing between 0% relative humidity and 50% relative humidity. In FIG. 9, on the other hand, the wavelength shift is only 30 pm. No hydrogen is used in these experiments. In this way, the unwanted response to water in the surroundings is reduced. Also, the response time when the reaction layer is exposed to hydrogen is reduced in the hydrophobic polyimide (not shown).

The embodiment of FIG. 8 can still be used as a hydrogen sensor in air with humidity, since a reference fiber without a reaction layer, but coated with just polyimide can be used. In this way, the effect of the water absorption in the hydrogen sensor can be compensated for. This can also still be done for the embodiment of FIG. 9, wherein hydrophobic polyimide is used, since there is still a response when the sensor is exposed to humid air.

FIG. 10 shows an embodiment of the sensing assembly 1000 wherein an optical fiber 1006 is coated along its entire length with the reaction layer comprising polyimide, manufactured using techniques such as a draw tower, such that a distributed hydrogen sensor is created, wherein the hydrogen concentration can be measured along the full length, or at least the coated length, of the fiber. This enables the manufacturing of long lengths of the sensing assemblies described above, thereby enabling measuring of hydrogen concentration in the surroundings in a continuous manner along the fiber length. In this case, the temperature sensitive detection zone can be as long as the length of fiber coated with the reaction layer, for instance 1 m, 2 m, or along the full length of the fiber.

The distributed hydrogen sensor functions according to the principles of distributed temperature sensing, further including correlating the measured temperature to a certain hydrogen concentration. Here, a laser source 1002 emits laser pulses 1008 into the fiber core of the fiber 1006. At a certain point 1012 in the fiber 1006, there is an imperfection at which the laser pulse scatters in various ways, including Rayleigh scattering, Raman Stokes scattering and Raman anti-Stokes scattering. The latter anti-Stokes scattering has a wavelength shift towards wavelengths below the wavelength of the laser pulse 1008, and its intensity is temperature dependent. A part of the scattered light is back scattered light 1010. The anti-Stokes back scattered light is detected by detector 1004. The position of point 1012 can be determined by taking into account the time-of-flight of the laser pulse 1008. Putting these data together, the temperature at point 1012 is determined. Since the temperature change associated with a certain is fixed for specific used reactive layers and reactive compositions as described above, the hydrogen concentration at point 1012 can then be determined by the distributed hydrogen sensor.

Here, for sake of explaining the distributed hydrogen sensor, only one point 1012 is shown. Imperfections in the fiber core and/or fiber core/fiber cladding interface cause (elastic and non-elastic) scattering along the full length of the optical fiber. Therefore, along the whole length of the optical fiber, a plurality of points exist and therefore a temperature profile of the whole optical fiber, and thus a hydrogen concentration profile for the coated part of the fiber, can be obtained.

Although a distributed hydrogen sensor is described above, it will be apparent that in the same way, a distributed sensor for different reactive gases can be made.

### Example of Preparing the Reactive Layers

Nano-sized tungsten oxide powder was prepared using a sol-gel method. An aqueous sol and gel of tungstic acid (H₂WO₄) were prepared from Na₂WO₄ with protonated cation-exchange resin. The nano-tungsten oxide in its hydrate form WO₃—H₂O is finally obtained by repeatedly washing with distilled water and centrifuging of the gel. Appropriate amounts of hexachloroplatinum (H₂PtCl₆) solution (atomic ratio WO₃:Pt of around 10:1) were added to synthesized WO₃ in order to obtain the reaction layer used for hydrogen detection. After annealing at 400° C. during 2 hours, a powder consisting essentially of nanosized tungsten oxide nanoplatelets is obtained. In general, annealing at the curing temperature (of at least 180° C.) for a minimum of 1 hour is preferred. The platinum is dispersed on the surface of these platelets.

The production of FBG's in optical fibers is described in numerous sources. For example, the section “Example” in WO 2008/125686 A1, starting on page 13, last paragraph, can be used to obtain uniform FBG's that can be used in the sensor assemblies of the present application.

Prior to the deposition of the reaction layer, the optical fiber was cleaned. The reaction layer without polyimide was then applied to the optionally stripped optical fiber at a place where an FBG was written. The powder of tungsten oxide doped with platinum was mixed with a solvent so that the optical fiber could be immersed into the solution. Then the solvent was evaporated at ambient temperature and the reaction layer remained fixed on the optical fiber. The thickness of the deposited layer was measured to be of the order of several microns.

As shown in FIG. 5, for a 2% concentration of H₂ in dry air, the measured wavelength shift is equal to about 4 nm (4000 pm). It is equivalent to an increase of temperature around the FBG of about 400° C. since the temperature sensitivity of the FBG is of the order of 10 pm/° C. At room temperature, the response time is about 2 s.

The reaction layer containing porous polyimide (PI) was prepared by mixing appropriate amounts of the nano-sized WO₃:Pt powder, PI precursor (40 wt % relative to WO₃:Pt) and the porogen PEG (low molecular mass M_{w}=300 g/mol, around 50 wt % relative to WO₃:Pt). When a (more) hydrophobic reaction layer is wanted, hydrophobic monomers (40 wt. % relative to the PI precursor) are added to the PI precursor prior to adding the other components. The PI-mix is cured at 250° C. for 1 hour, whereby the PEG decomposes and leaves behind a porous structure of PI—WO₃:Pt. The curing can also be done in multiple steps, or at different temperatures and times, depending on the required porosity, the material properties of the porogon and/or the polyimide based polymer material.

The fiber is optionally pretreated with an adhesion promoter resulting in an improved bond between the reaction layer and the optical fiber. The adhesion promoter may be an organosilane that comprises either an amino group, for example aminosilane-based adhesion promoters such as a aminopropyltrimethoxysilane. Other organosilanes with different functional groups may also be used as adhesion promoters.

Other materials functioning as porogen include polyvinylpyrrolidone and polypropylene. For instance, polyvinylpyrrolidone with a molecular weight M_{w}<=10000 Da has a suitable decomposition temperature of <=175° C., viz. lower than the curing temperature of the used polyimide. More generally, a porogen can be any material that doesn't react with the PI components and have a lower decomposition temperature than the curing temperature of PI. Alternatively, materials that withstand the curing process and remain in the coating during curing, but are soluble in a solvent that does not dissolve the PI—WO₃:Pt can be used as porogen. These latter materials can be washed out after curing with the solvent.

The optical fibers can be coated with a mixture of polyimide and the reactive composition in various ways, such as dip coating with a mixture of a polyimide-based polymer solution with reactive composition and curing thereafter. This may be repeated as needed to obtain the required thickness. It is possible to first coat the fiber with polyimide-based polymer without reactive composition, while only an outer layer is coated with the combination of polyimide-based polymer and reactive composition. One advantage is that a fiber draw tower can be used to manufacture long lengths of the sensing assemblies described above, enabling measuring of hydrogen concentration in the surroundings in a continuous manner along the fiber length.

A smaller scale alternative for applying is a polyimide recoater, such as the Vytran polyimide fiber recoater.

A fiber draw tower configured for preparing a fiber core and coating that fiber core with polyimide can be used. One example of a suitable draw tower is the OFC 20SF of Rosendahl Nextrom, wherein after a dip coating step a step of heat curing is applied.

One way of preparing polyimide in a suitable manner is from polyamic acids (PAA) built of monomers selected from the group consisting of benzophenone tetracarboxylic dianhydrides, 4,4-oxydianilines, phenylenediamines, or byphenyldianhydrides, pheylenediamines, or combinations thereof, preferably poly(3,3′,4,4′-benzophenonetetracarboxylic dianhydride-co-4,4′-oxydianiline/1,3-phenylenediamine), amic acid solution. Additionally or alternatively, PAA of (3,3′,4,4′-biphenyltetracarboxylic dianhydride/1,4-phenylenediamine) in a suitable solvent, preferably N-methylpyrrolidinone, or other solvents such as in aprotic polar solvents e.g. N, N-dimethylacetamide, N, N-dimethylformamide, dimethyl sulfoxide, can be used. Examples of commercially available polyimide precursor material suitable for use as the polyimide based polymer material are PI2525 manufactured by HD Microsystems. This precursor has suitable coefficient of thermal expansion CTE (40 ppm), moisture uptake (2-3) %, flexibility, adhesion to silica surface, tensile strength and modulus of about 2.5 GPa. Other options include PI2555, PI2610, and PI2611 of HD Microsystems, having lower moisture uptake than PI2525, or PI 2545 and PI5878G, which have a higher thermal stability than PI2525.

Polyimide films containing ester-linkages in the polymer backbones (poly(ester-imide) s, e.g. bis(trimellitic acid anhydride)phenyl ester, or PI from other aromatic dianhydrides and aromatic diamines, or from sulfonated diamines with suitable characteristics may also be used as polymer matrix material.

The disclosure comprises the following clauses:

1. Sensing assembly for detecting a reactive gas to be sensed such as hydrogen, the sensing
assembly being for use in a gas sensor, the sensing assembly comprising
an optical fiber having a temperature sensitive detection zone;
a reaction layer associated with an external surface of the optical fiber positioned at least adjacent to the temperature sensitive detection zone, wherein the reaction layer comprises a polyimide-based polymer and a reactive composition adapted to generate a temperature change when brought into contact with the gas to be sensed.

2. Sensing assembly according to clause 1, wherein the polyimide-based polymer causes a reduced reaction rate at the reactive composition of the reactive gas to be sensed.

3. Sensing assembly according to clause 2, wherein the polyimide-based polymer is a polyimide-based polymer matrix material comprising the reactive composition.

4. Sensing assembly according to any one of clauses 1 to 3, wherein the polyimide-based polymer comprises hydrophobic monomers, wherein preferably the polyimide-based polymer comprises 10-80 wt % of hydrophobic monomers relative to polyimide, more preferably 20-40 wt %.

5. Sensing assembly according to clause 4, wherein the hydrophobic monomers are hydrophobic fluorinated monomers preferably selected from the group consisting of fluorinated diamines or fluorinated dianhydrides, more preferably selected from the group consisting of 2,2-bis(3-amino-4-hydroxyphenyl) hexafluoropropane, 2,2-bis(3,4-dicarboxyphenyl) hexafluoropropane dianhydride, 3,4-dicarboxy-1,2,3,4-tetrahydro-6-fluoro-1-naphthalene succinic dianhydride, bis(trifluoromethyl)benzidine, or 1,4-bis(4-amino-2-trifluoromethylphenoxy)benzene, bis(2-trifluoromethyl-4-aminophenyl) terephthalat and a combination thereof.

6. Sensing assembly according to any one of clause 1-5, wherein the reaction layer is porous.

7. Sensing assembly according to any one of the preceding clauses, wherein the reactive composition comprises tungsten oxide or vanadium oxide, and a catalyst selected from the group consisting of catalytic metals and catalytic metal oxides, preferably the catalytic metals comprise platina.

8. Sensing assembly according to any one of the preceding clauses, wherein the temperature sensitive detection zone comprises a fiber Bragg grating.

9. Sensing assembly according to any one of the preceding clauses, wherein the optical fiber comprises a fiber core, a fiber cladding surrounding the fiber core and a fiber coating surrounding the fiber cladding, wherein the fiber core and fiber cladding comprise the temperature sensitive detection zone, wherein at least an outer part of the fiber coating comprises the reaction layer.

10. Sensing assembly according to clause 9, wherein the temperature sensitive zone is comprised by the fiber core and fiber cladding.

11. Sensing assembly according to the previous clause, wherein the fiber core is configured for distributed temperature sensing, DTS, wherein the gas sensor is a DTS-based gas sensor.

12. Sensing assembly according to clause 10 or 11, wherein the optical fiber comprises the reaction layer continuously along at least a length of 0.3 m, preferably at least 1 meter.

13. Sensing assembly according to any one of the preceding clauses, wherein the reactive composition is adapted to generate heat when brought into contact with the gas to be sensed.

14. Sensing assembly according to one of the preceding clauses, wherein the reactive gas to be sensed is hydrogen.

15. Sensing assembly according to one of the preceding clauses, wherein
the sensing assembly comprises a plurality of optical fibers and corresponding reaction layers; and/or
wherein the optical fiber comprises a plurality of temperature sensitive detection zones.

16. Method of manufacturing a sensing assembly for detecting a reactive gas to be sensed such as hydrogen, the sensing assembly being for use in a gas sensor, the method comprising
providing an optical fiber having a temperature sensitive detection zone;
applying a reaction layer onto an external surface of the optical fiber, wherein the reaction layer is applied at least adjacent to the temperature sensitive detection zone, wherein the reaction layer comprises a polyimide-based polymer and a reactive composition adapted to generate a temperature change when brought into contact with the gas to be sensed.

17. Method according to clause 16, wherein the applying of the reaction layer comprises
providing a mixture comprising a polyimide precursor, the reactive composition and a sacrificial compound decomposing at a decomposing temperature and
applying the mixture onto the external surface of the optical fiber, wherein the reaction layer is applied at least adjacent to the temperature sensitive detection zone,
wherein the method comprises, after applying the mixture, annealing the sensing assembly at elevated temperature of at least the decomposing temperature such that the reaction layer is formed, wherein the formed reaction layer is porous.

18. Method according to clause 17, wherein the sacrificial compound is selected from the group consisting of polyether compounds, polyvinylpyrrolidone and polypropylene, wherein preferably the sacrificial compound is a polyether compound, more preferably wherein the polyether compound is polyethylene glycol, PEG.

19. Sensing assembly manufactured according to the method of any one of clauses 16-18.

20. Gas sensor comprising the sensing assembly according to any one of clauses 1-15 and clause 19.

21. Gas sensor according to the previous clause, wherein the gas sensor is a hydrogen gas sensor.

Whilst the principles of the described assemblies, methods and sensors, devices have been set out above in connection with specific embodiments, it is to be understood that this description is merely made by way of example and not as a limitation of the scope of protection which is determined by the appended claims.

1. A sensing assembly for detecting a reactive gas to be sensed such as hydrogen, the sensing assembly being for use in a gas sensor, the sensing assembly comprising:
an optical fiber having a temperature sensitive detection zone; and
a reaction layer associated with an external surface of the optical fiber positioned at least adjacent to the temperature sensitive detection zone, wherein the reaction layer comprises a polyimide-based polymer and a reactive composition adapted to generate a temperature change when brought into contact with the gas to be sensed.

2. The sensing assembly according to claim 1, wherein the polyimide-based polymer causes a reduced reaction rate at the reactive composition of the reactive gas to be sensed.

3. The sensing assembly according to claim 2, wherein the polyimide-based polymer is a polyimide-based polymer matrix material comprising the reactive composition.

4. The sensing assembly according to claim 1, wherein the polyimide-based polymer comprises hydrophobic monomers.

5. The sensing assembly according to claim 4, wherein the hydrophobic monomers are hydrophobic fluorinated monomers.

6. The sensing assembly according to claim 1, wherein the reaction layer is porous.

7. The sensing assembly according to claim 1, wherein the reactive composition comprises tungsten oxide or vanadium oxide, and a catalyst selected from the group consisting of catalytic metals and catalytic metal oxides.

8. The sensing assembly according to claim 1, wherein the temperature sensitive detection zone comprises a fiber Bragg grating.

9. The sensing assembly according to claim 1, wherein the optical fiber comprises a fiber core, a fiber cladding surrounding the fiber core and a fiber coating surrounding the fiber cladding, wherein the fiber core and fiber cladding comprise the temperature sensitive detection zone, wherein at least an outer part of the fiber coating comprises the reaction layer.

10. The sensing assembly according to claim 9, wherein the temperature sensitive zone is comprised by the fiber core and fiber cladding.

11. The sensing assembly according to claim 10, wherein the fiber core is configured for distributed temperature sensing, DTS, wherein the gas sensor is a DTS-based gas sensor.

12. The sensing assembly according to claim 10, wherein the optical fiber comprises the reaction layer continuously along at least a length of 0.3 m, preferably at least 1 meter.

13. The sensing assembly according to claim 1, wherein the reactive composition is adapted to generate heat when brought into contact with the gas to be sensed.

14. The sensing assembly according to claim 1, wherein the reactive gas to be sensed is hydrogen.

15. The sensing assembly according to claim 1, wherein
the sensing assembly comprises a plurality of optical fibers and corresponding reaction layers; and/or
wherein the optical fiber comprises a plurality of temperature sensitive detection zones.

16. A method of manufacturing a sensing assembly for detecting a reactive gas to be sensed such as hydrogen, the sensing assembly being for use in a gas sensor, the method comprising:
providing an optical fiber having a temperature sensitive detection zone; and
applying a reaction layer onto an external surface of the optical fiber, wherein the reaction layer is applied at least adjacent to the temperature sensitive detection zone, wherein the reaction layer comprises a polyimide-based polymer and a reactive composition adapted to generate a temperature change when brought into contact with the gas to be sensed.

17. The method according to claim 16, wherein the applying of the reaction layer comprises:
providing a mixture comprising a polyimide precursor, the reactive composition and a sacrificial compound decomposing at a decomposing temperature; and
applying the mixture onto the external surface of the optical fiber, wherein the reaction layer is applied at least adjacent to the temperature sensitive detection zone,
wherein the method comprises, after applying the mixture, annealing the sensing assembly at elevated temperature of at least the decomposing temperature such that the reaction layer is formed, wherein the formed reaction layer is porous.

18. The method according to claim 17, wherein the sacrificial compound is selected from the group consisting of polyether compounds, polyvinylpyrrolidone and polypropylene.

19. A sensing assembly manufactured according to the method of claim 16.

20. A gas sensor comprising:
an optical fiber having a temperature sensitive detection zone; and
a reaction layer associated with an external surface of the optical fiber positioned at least adjacent to the temperature sensitive detection zone, wherein the reaction layer comprises a polyimide-based polymer and a reactive composition adapted to generate a temperature change when brought into contact with the gas to be sensed;
wherein the sensing assembly is manufactured according to the method of claim 16.

21. (canceled)
